(19) United States
(12) Patent Application Publication
(10) Pub. No.: US 2017/0007617 A1
(43) Pub. Date: Jan. 12, 2017

(54) INTRAVENOUS FORMULATIONS OF A LATE SODIUM CURRENT INHIBITOR

(71) Applicant: Gilead Sciences Drive, Foster City, CA (US)

(72) Inventor: Robert G. Strickley, San Mateo, CA (US)

(21) Appl. No.: 15/202,877

(22) Filed: Jul. 6, 2016

Related U.S. Application Data
(60) Provisional application No. 62/190,430, filed on Jul. 9, 2015.

(51) Int. Cl. A61K 31/553; A61K 9/00; A61K 47/48

(52) U.S. Cl. CPC A61K 31/553; A61K 47/48969; A61K 9/0019

(57) ABSTRACT

An intravenous pharmaceutical composition or kit comprising 4-(pyrimidin-2-ylmethyl)-7-(4-(trifluoromethoxy)phenyl)-3,4-dihydrobenzo[f][1,4]oxazepin-5(2H)-one (Compound I) and a beta-cyclodextrin derivative.

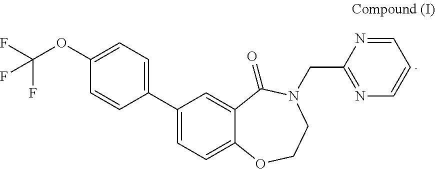 [SMILES: O=C1c2cc(-c3ccc(OC(F)(F)F)cc3)ccc2OCCN1Cc1ncccn1]

### CROSS REFERENCE TO RELATED APPLICATIONS

This application claims the benefit under 35 U.S.C. §119(e) to U.S. Provisional Application Ser. No. 61/190,430, filed Jul. 9, 2015 the entirety of which is incorporated herein by reference.

### BACKGROUND

The present disclosure relates to intravenous formulations of the late sodium current (INaL) inhibitor compound 4-(pyrimidin-2-ylmethyl)-7-(4-(trifluoromethoxy)phenyl)-3,4-dihydrobenzo[f][1,4]oxazepin-5 (2H)-one.

Late sodium current (INaL) is a sustained component of the fast Na⁺ current of cardiac myocytes and neurons. Certain neurological and cardiac conditions are associated with abnormal INaL enhancement which contributes to the pathogenesis of both electrical and contractile dysfunction in mammals. See, for example, Pathophysiology and Pharmacology of the Cardiac “Late Sodium Current”, Pharmacology and Therapeutics 119 (2008), 326-339. Accordingly, compounds that selectively inhibit INaL in mammals may be useful in treating such cardiovascular and neuronal disease states. Such cardiovascular disease states include, for example, atrial fibrillation, long QT syndrome, and hypertrophic cardiomyopathy.

The compound 4-(pyrimidin-2-ylmethyl)-7-(4-(trifluoromethoxy)phenyl)-3,4-dihydrobenzo[f][1,4]oxazepin-5(2H)-one, designated herein as Compound (I), is known to be a selective late sodium current inhibitor as described, for example, in WO 2013/006485. There is a need to administer Compound I intravenously to certain patients. However, Compound (I) is relatively insoluble in aqueous media. Thus, there is a need to develop an intravenous (IV) formulation of Compound I that exhibits improved solubility and/or improved usability for IV administration compared to other options of IV formulations.

### SUMMARY

The present disclosure provides intravenous (IV) formulations comprising 4-(pyrimidin-2-ylmethyl)-7-(4-(trifluoromethoxy)phenyl)-3,4-dihydrobenzo[f][1,4]oxazepin-5(2H)-one, a beta-cyclodextrin derivative, and a pharmaceutically acceptable excipient or carrier.

The disclosure provides an intravenous pharmaceutical composition comprising Compound (I)

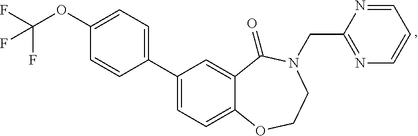 [SMILES: O=C1c2cc(-c3ccc(OC(F)(F)F)cc3)ccc2OCCN1Cc1ncccn1]

a beta-cyclodextrin derivative, a pharmaceutically acceptable excipient or carrier.

In one embodiment, the beta-cyclodextrin derivative is Captisol®. Captisol® is represented by the formula

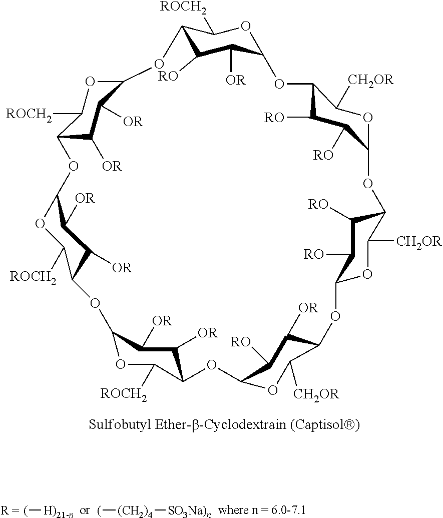

In one embodiment, the beta cyclodextrin derivative is Dexolve-7®.

In one embodiment, the beta cyclodextrin derivative is Cavitron®.

In one embodiment, the beta cyclodextrin derivative is Kleptose®.

In one embodiment, the disclosure provides an intravenous formulation comprising Compound (I), a beta cyclodextrin derivative, a pharmaceutically acceptable excipient or carrier useful for the treatment of cardiovascular diseases.

In another embodiment, the disclosure provides an intravenous formulation comprising Compound (I) and Captisol® or an intravenous formulation comprising Compound (I) and Dexolve-7® each with a pharmaceutically acceptable excipient or carrier

In another embodiment, the present disclosure provides a method of treating a cardiovascular disease comprising administering an intravenous composition comprising Compound (I), a beta-cyclodextrin derivative and a pharmaceutically acceptable excipient or carrier, to a human patient in need thereof.

In another embodiment, the present disclosure provides a method of treating a cardiovascular disease selected from atrial fibrillation, ventricular tachycardia, ventricular fibrillation, LQT syndromes, heart failure, and hypertrophic cardiomyopathy comprising administering an intravenous composition comprising Compound (I), a beta-cyclodextrin derivative and a pharmaceutically acceptable excipient or carrier, to a human patient in need thereof.

In another embodiment, the present disclosure provides a method of treating a cardiovascular disease comprising administering an intravenous composition comprising Compound (I) and a beta-cyclodextrin derivative selected from the group consisting of Captisol®, Dexolve-7®, Cavitron®, and Kleptose®; and a pharmaceutically acceptable excipient or carrier to a human patient in need thereof.

In another embodiment, the present disclosure provides the use of an intravenous composition comprising Compound (I), a beta-cyclodextrin derivative and a pharmaceutically acceptable excipient or carrier for the manufacture of a medicament for the treatment of cardiovascular diseases.

In another embodiment, the present disclosure provides an intravenous composition comprising Compound (I) and a beta-cyclodextrin derivative selected from the group consisting of Captisol®, Dexolve-7®, Cavitron®, and Kleptose®; and a pharmaceutically acceptable excipient or carrier for the manufacture of a medicament for the treatment of cardiovascular diseases.

In another embodiment, the present disclosure provides for the use of an intravenous composition comprising Compound (I) and a cyclodextrin derivative for the manufacture of a medicament for the treatment of cardiovascular diseases selected from atrial fibrillation, ventricular tachycardia, ventricular fibrillation, LQT syndromes, heart failure, and hypertrophic cardiomyopathy.

### DEFINITIONS

Captisol® is a registered trademark of Ligand Corporation. Captisol® refers to beta cyclodextrin sold by or licensed by Ligand Pharmaceuticals. Further characteristics of Captisol® are described herein.

TWEEN 80® is a registered trademark of Sigma-Aldrich Company. It is a form of polysorbate, a non-ionic detergent/surfactant used for selective protein extraction and isolation of nuclei from mammalian cell lines.

Dexolve-7® is a registered trademark of CycloLabs Limited. Dexolve 7® is sulfobutylalkyl ether beta cyclodextrin sodium salt, an excipient used in pharmaceutical formulations to improve solubility. See also, http://ujoldal.cyclolab.hu/images/pdf/DexolveCycloLab3.pdf.

Cavitron® is a registered trademark of Wacker Chemie AG. Cavitron is an excipient obtained by the substitution of hydroxyl groups on native cyclodextrins to make hydroxypropyl-β-cyclodextrins (HPBCD), a process that significantly enhance their solubility and makes them more suitable for drug solubilization.

Kleptose® is a registered trademark of Roquette Pharmaceuticals, Geneva, Ill., USA. Kleptose® is a brand of hydroxypropyl beta-cyclodextrin.

Beta cyclodextrins are 7-membered sugar ring molecules or derivatives used for solubilizing hydrophobic drugs in medicinal agents for enhanced solubility of said hydrophobic drugs when administered to a patient. When used for oral or non-IV applications they may also provide enhanced bioavailability of the drug to the patient. As used herein the phrase “beta cyclodextrin derivative” refers to one or more of Captisol®, Dexolve®, Cavitron®, Kleptose® and the like.

The acronym “SBE” as used herein denotes sulfobutylalkyl beta cyclodextrin.

V-Fend® is a registered trademark of Pfizer Corporation. V-Fend is an SBE beta cyclodextrin-enabled formulation of voriconazole.

Nexterone® is a registered trademark of Baxter Corporation. It represents a beta cyclodextrin-enabled IV formulation of amiodarone.

The term “treatment” or “treating” means any administration of Compound I according to the present disclosure to a subject (e.g. human) having or susceptible to a condition or disease disclosed herein for the purpose of: 1) preventing or protecting against the disease or condition, that is, causing the clinical symptoms not to develop; 2) inhibiting the disease or condition, that is, arresting or suppressing the development of clinical symptoms; or 3) relieving the disease or condition that is causing the regression of clinical symptoms. In some embodiments, the term “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent e.g. Compound I, to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition.

### DETAILED DESCRIPTION

The compound 4-(pyrimidin-2-ylmethyl)-7-(4-(trifluoromethoxy)phenyl)-3,4-dihydrobenzo[f][1,4]oxazepin-5(2H)-one (Compound (I)) is a selective and potent late sodium current inhibitor.

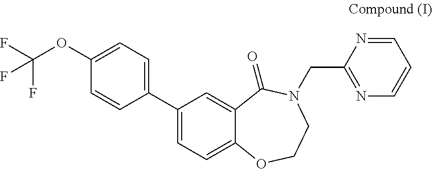 [SMILES: O=C1c2cc(-c3ccc(OC(F)(F)F)cc3)ccc2OCCN1Cc1ncccn1]

In another embodiment, the present disclosure provides a method of treating a cardiovascular disease comprising administering an intravenous composition comprising Compound (I), a beta-cyclodextrin derivative and a pharmaceutically acceptable excipient or carrier to a human patient in need thereof.

The present disclosure results from the surprising discovery that an intravenous formulation comprising a beta cyclodextrin derivative improves the ability to administer Compound (I) to a human patient in need thereof without the side effects and/or practical issues such as excessive volumes, low solubility and attendant potential for precipitation, excessive organic phase, etc., associated with other solubility enhancing agents. Such practical issues may in some cases (e.g. precipitation) translate to issues of safety and efficacy.

The Compound (I) has low water solubility (0.01 mg/mL) relative to an anticipated human efficacious dose of about 1-300 mg, preferably about preferably 1-60 mg. Therefore, a solubilization technique is needed for an effective and practical intravenous formulation. It is desired to administer Compound (I) by intravenous infusion after the formulation is diluted (preferably to about 50 mL or 100 mL) in saline solution. Compound (I) is chemically stable in solution thus an intravenous formulation is practicable. Intravenous formulation is also desired from a clinical perspective to administer to a patient in a hospital type setting to achieve quick onset of effect or for the treatment of a patient who cannot be administered an oral formulation of Compound (I). Certain solubilization techniques are known to one of ordinary skill in the art. Prior to applicants' research however, it is not known which, if any, of many solubilization enhancing techniques will prove effective in view of the desired clinical parameters, considerations of effective concentrations, effective infusion rates, safety of operation and/or delivery devices, etc.

Applicants have discovered that beta-cyclodextrin derivatives serve as effective solubilizing agents that achieve the desired goals of providing effective IV formulations of Compound (I) and avoid shortcomings of alternative solubilizing agents. Examples of solubilizing agents are provided below.

It is desired to develop a ready-to-use intravenous formulation of compound (I) and/or an intravenous formulation of compound (I) that requires dilution prior to use. A ready-to-use intravenous formulation could be either a large volume parenteral (LVP) for infusion-only or a small volume parenteral (SVP) for bolus use or diluted prior to infusion. A preferred ready-to-use LVP intravenous formulation for infusion-only would be aqueous-based; relatively dilute in the concentration of Compound (I) (e.g. <1.2 mg/mL for clinical); and would need to be packaged in an infusion bag. A ready-to-use SVP intravenous formulation could be administered by either as a bolus or diluted prior to infusion. Such SVP intravenous formulation could be packaged in standard glass vials and stoppers. Such SVP intravenous formulation would also be aqueous-based but with a higher concentration of Compound (I) (i.e. ≧2.5 mg/mL) than an LVP intravenous formulation or other intravenous formulations. A SVP intravenous formulation could also be an organic solvent-based pre-concentrate with a relatively high concentration of Compound (I) (˜25 mg/mL). Such formulation would require dilution prior to administration by infusion and could be packaged in standard glass vials but would likely require special stoppers.

### TWEEN 80®

TWEEN 80® is a surfactant excipient (Formula III) that is used in pharmaceutical injectable formulation to either increase solubility or prevent surface adsorption.

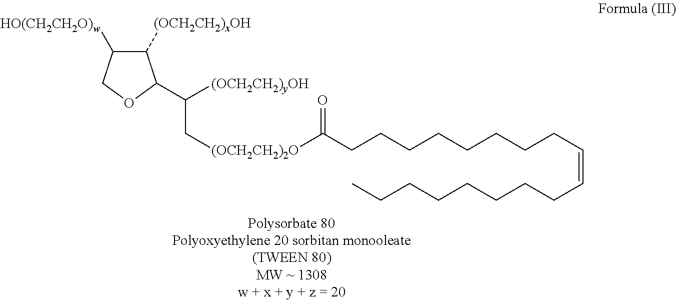

TWEEN 80® is commonly used at low levels (0.001-0.1%) in biomolecule (proteins and antibodies) formulations to prevent surface adsorption. At higher levels (≧8%) TWEEN 80 can solubilize some small molecule drugs and is currently in use with at least four FDA-approved injectable medications of small molecules. A major concern with the use of TWEEN 80® is the potential for hemodynamic effect. (See E J Kronski, Hemodynamic Effects of Intravenous Amiodarone, JACC, 4(3):565-570 (1984)), and pharmacological effects (see A J Ten Tije, J Verweij, W J Loos, A Sparreboom, Pharmacological Effects of Formulation Vehicles, Clin Pharmacokinet, 42(7): 665-685 (2003)). In addition, there are several other potentially significant issues with the use of TWEEN 80® in intravenous formulations including:
1) Potential hemodynamic effect
2) Tubing limitations
3) Extractables and leachables from tubing
4) Limitations on stoppers
5) Potential for precipitation upon dilution
6) Adaptability for pediatric use

### CAPTISOL®

Captisol® is an excipient that has been used in certain pharmaceutical injectable formulations to increase solubility. Captisol® is a water-soluble cyclic molecule and is a sulfobutylether-β-cyclodextrin (Formula II) derivative. Captisol® is a donut shaped complexing agent in which small molecules can be solubilized by incorporation into the donut-like cavity. The on-off rate of the complex is rapid and does not affect the pharmacokinetics of the compound solubilized. Captisol® is safe and is currently used in certain FDA-approved human injectable medications.

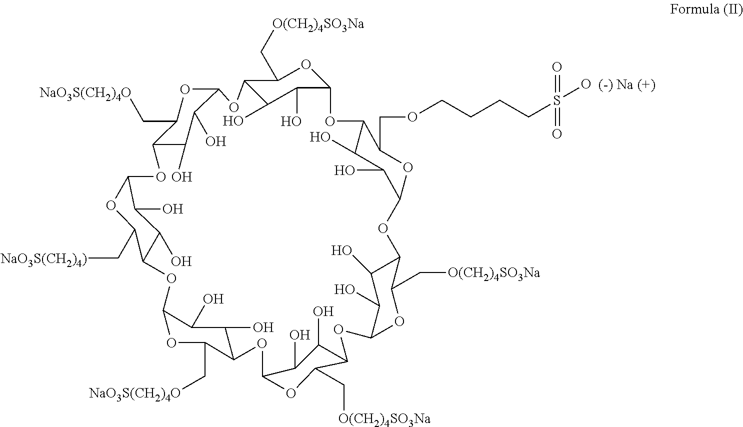

The chemical structure of Captisol®, sulfobutylether-β-cyclodextrin Captisol® is shown above. The composition of Captisol®, certain methods of using Captisol® in formulations, and methods and/or processes for its manufacture are disclosed variously in U.S. patents U.S. Pat. No. 5,134,127, U.S. Pat. No. 5,376,645, U.S. Pat. No. 7,635,773, U.S. Pat. No. 7,625,878, and U.S. Pat. No. 8,410,077 the entirety of each of which is incorporated herein by reference.

A Captisol®-containing intravenous formulation of Compound (I) may be developed using an amount of Captisol® administered that is below the level in current applications. Given the specifications or co concentrations herein, one of ordinary skill in the art is able to develop Captisol® formulation for intravenous or other uses. Developing a Captisol®-containing intravenous formulation would provide significant benefits over a TWEEN 80®-containing formulation and would minimize and eliminate many of the potential safety and pharmacological risks with the use of TWEEN 80® as well as the handling issues discussed previously.

A potential clinical and commercial packaging configuration for a 2.5 mg/mL intravenous formulation of Compound (I) composed of water with 12% Captisol® is 8 mL in a 10-mL glass vial with standard stoppers and contain about 20 mg of Compound (I). Thus, the use of Captisol® or equivalent sulfobutylalkyl beta cyclodextrin formulation of Compound (I) is optimum at enhancing solubility of Compound (I), avoiding the risks associated with using TWEEN 80® and avoids the practical issues of excessive organic phase, excessive transfusion or dilution volumes, and other issues outlined above.

### Aqueous

The water solubility of the compound of formula (I) is low at about 0.01 mg/mL. Therefore, a purely aqueous formulation is neither optimally useful nor practicable and fails to meet an objective of dissolving 1-300 mg of Compound (I) for use in the treatment of diseases disclosed herein. Thus, an effective solubilization technique is needed for a successful intravenous formulation.

### Co-Solvents

Co-solvents of water-soluble excipients and water were explored without success. In a co-solvent system the drug solubility decreases logarithmically as the percent of solvent decreases linearly, thus there is a potential to precipitate upon dilution. To achieve the 0.3 mg/mL needed in the infusion media for a 15 mg dose of Compound (I), the amount of solvent required in a PEG 400/water co-solvent system is more than 30% PEG 400. However, this is not feasible due to the large amount of organic solvent that would be injected (>15 mL in a 50 mL infusion). Therefore, a co-solvent solubilizing technique is insufficient to achieve the concentrations of Compound (I) required for either a SVP or a LVP.

### Co-Solvents and a Surfactant

Co-solvents of a water soluble excipient and a surfactant (e.g. TWEEN 80®) in water were explored but without success. For example, to achieve a 0.3 mg/mL concentration needed in the infusion media for a 15 mg dose of Compound (I), the amount of solvent and surfactant required is about 0.4% TWEEN 80® and about 0.6% PEG 400 in water, in which the solubility of Compound (I) is 0.4 mg/mL. To achieve the 1.2 mg/mL needed in the infusion media for a 60 mg dose of Compound (I), the amount of solvent and surfactant required is 1.6% TWEEN 80 and 2.4% PEG 400 in water, in which Compound (I) solubility is 1.4 mg/mL. These data suggest that an aqueous-based LVP is possible, but there would be significant limitations and issues of compatibility of the formulation with the inner surface of the infusion bag due to the surfactant TWEEN 80®.

### Pre-Concentrate

Pre-concentrated formulations of a surfactant and an organic solvent (pre-concentrates) were explored. Data in the section on Co-solvents and a Surfactant suggests that a pre-concentrated formulation of 40% TWEEN 80 and 60% PEG 400 is a commercially-viable formulation strategy but the concentration of Compound (I) would need to be ≦30 mg/mL in order to avoid precipitation upon dilution. Solubility of Compound (I) in 40% TWEEN 80 and 60% PEG 400 is greater than 100 mg/mL. Therefore, a 25 mg/mL of Compound (I) pre-concentrate in 40% TWEEN 80® and 60% PEG 400 is a commercially-viable intravenous formulation from a solubility perspective, but would only be suitable for dilution prior to intravenous infusion. Prototypes of this pre-concentrate formulation have been made and shown to not precipitate upon dilution into saline. However, there remain the problems associated with the use of TWEEN 80® as disclosed previously.

### Complexation

Complexation with a beta-cyclodextrin derivative was explored successfully. In a solution that utilizes complexation with beta cyclodextrin derivative, the drug solubility decreases linearly (assuming a 1:1 complex) as the amount of cyclodextrin decreases linearly. Thus, there is no potential for precipitation of Compound I upon dilution. The two beta cyclodextrin derivatives that have been used most in commercial injectable formulations are sulfobutyl alky ether and alkyl ether beta cyclodextrin derivative e.g. Captisol® and hydroxypropyl-β-cyclodextrin derivative e.g. Kleptose® respectively. A formulation of water with 12% w/w Captisol® is isotonic. The solubility of Compound (I) in water with 3.6%, 12% and 36% Captisol® is 1.1, 3.9 and 13.7 mg/mL respectively. A formulation of water with 28% hydroxypropyl-β-cyclodextrin is isotonic. The solubility of Compound (I) in water with 7%, 14% and 28% and hydroxypropyl-β-cyclodextrin is 3.2, 7.4 and 18 mg/mL respectively. Therefore, both Captisol® and hydroxypropyl-β-cyclodextrin can solubilize Compound (I). The use of Captisol® or Dexolve-7®—both sulfobutyl ether beta cyclodextrin derivatives, is preferred over the use of Kleptose®—a hydroxypropyl-β-cyclodextrin derivative. The use of hydroxypropyl-β-cyclodextrin as an excipient in intravenous formulations has been limited. Certain commercial products incorporating hydroxypropyl-β-cyclodextrin have been discontinued or withdrawn from the market. (See for example, http://www.fda.gov/Drugs/InformationOnDrugs/ucm091564.htm; (accessed Jul. 7, 2015)

Therefore, a 2.5 mg/mL concentration of Compound (I) formulation in water with 12% Captisol® or equivalent is a commercially-viable intravenous formulation from a solubility perspective, and in addition could be a ready-to-use bolus drug product or a pre-concentrate for dilution prior to infusion.

### Triglycerides, Oil-in-Water Emulsion

Compound (I) is soluble in triglycerides up to approximately 25 mg/mL in safflower oil. Thus in an oil-in-water emulsion with 20% oil the solubility would be ≦5 mg/mL. Therefore, an oil-in-water emulsion could potentially be developed, but the manufacturing and sterilization process is challenging as well as the physical stability of the biphasic emulsion. Therefore, an oil-in-water intravenous solution is not a desirable option for preparing an IV formulation of Compound (I).

### TWEEN 80®

1) A TWEEN/PEG intravenous formulation of Compound (I) may be developed and commercialized. However, safety and pharmacological risks exist with the use of TWEEN 80 (see Kronski E. Z J, et al, Hemodynamic Effects of Intravenous Amiodarone, JACC, 4(3):565-570 (1984) and A J ten Tije, J Verweij, W J Loos, A Sparreboom, Pharmacological Effects of Formulation Vehicles, Clin Pharmacokinet, 42(7): 665-685 (2003)) in addition to the handling issues as disclosed previously. A potential clinical and commercial packaging configuration for a 25 mg/mL pre-concentrated intravenous formulation composed of 40% TWEEN 80® and 60% PEG 400 is 1 mL in a 2-mL glass vial would likely require special stoppers and contain 25 mg of Compound (I). However, TWEEN 80® is not a—referred solubilizing agent on account of the hemodynamic and practical issues outlined previously. Amiodarone is a pertinent commercial example of switching from a TWEEN 80®-containing formulation to a Captisol®-containing intravenous formulation. The recently approved Captisol®-containing Nexterone® was developed to address the concerns of the use of TWEEN 80® in the existing TWEEN 80®-containing Captisol®. (See, D. J. Cushing, et. al.; Bioequivalence of 2 Intravenous Amiodarone Formulations in Healthy Participants; J. Clin Pharmacol, 49, 407-5115 (2009).; D. J. Cushing, P. R. Kowey, W. D. Cooper, B. W. Massey, M. R. Gralinski, R. J. Lipicky; PM101: A Cyclodextrin-based Intravenous Formulation of Amiodarone Devoid of Adverse Hemodynamic Effects; Eur. J. Pharmacology, 607, 167-172 (2009); and J. C. Somberg, et. al.; Lack of a Hypotensive Effect with Rapid Administration of a New Aqueous Formulation of Intravenous Amiodarone; The Amer. J. Cardiology, 93 (March 1), 576-581 (2004).

### Method of Use

The use of beta cydodextrin derivatives in formulating certain medicinal agents to improve solubility, safety and other parameters has been disclosed in, for example, U.S. patents U.S. Pat. No. 5,134,127, U.S. Pat. No. 5,376,645, U.S. Pat. No. 7,635,773, U.S. Pat. No. 7,625,878, and U.S. Pat. No. 8,410,077 the entirety of each of which is incorporated herein by reference. Prior to applicants' disclosure, the difficulty of preparing an IV solution of Compound (I) was unknown. Prior to applicants' disclosure the type of solubilizing agent(s) useful to achieve an effective IV formulation comprising Compound (I) was unknown. Applicants have surprisingly discovered that the use of beta-cyclodextrin derivatives is useful to achieve an effective IV solution of Compound (I). An IV solution of Compound (I) comprising a beta cyclodextrin derivative provides advantages of (1) ability to deliver varying required doses of Compound (I); (2) ability to adjust infusion rate without precipitation of Compound (I); (3) ability to avoid the problems associated with the use of other IV formulations such as the use of TWEEN 80® or organic co-solvents.

As a result of the discovery disclosed herein, one of ordinary skill in the art is able to (1) prepare an IV solution of Compound (I) as a bolus solution (i.e. for direct use; or as a pre-concentrate for dilution prior to use in a human patient in need thereof. Thus, the present disclosure also provides the use a kit comprising Compound (I) and a beta cyclodextrin derivative in an IV formulation for use in a human patient in need thereof. The kit may comprise a premixed LVP or SVP bag comprising Compound (I) and a beta cyclodextrin derivative. Alternatively, the kit may comprise a saline solution of a beta cyclodextrin (e.g. Captisol® or Dexolve-7®) and a vial of Compound (I) to be mixed on site or prior to use by one of ordinary skill in the art. The solution comprising a saline solution of a beta cyclodextrin e.g. Captisol® may need to be further diluted (e.g. use of about a 20-50% concentrate) by one of ordinary skill in the art prior to mixing to form an intravenous formulation for administration to a patient in need thereof. The present disclosure is thus directed to the preparation, manufacture and/or use of an intravenous formulation comprising Compound (I) and a beta cyclodextrin derivative for use in IV treatment. A preferred beta cyclodextrin derivative is Captisol®. Also preferred is the beta cyclodextrin derivative Dexolve-7®. Thus, in one embodiment, the present disclosure provides a kit comprising Compound (I) and a beta-cyclodextrin derivative for the treatment of cardiovascular diseases in a patient in need thereof. The ultimate decision on dosing rate, concentration of beta cyclodextrin derivative solution to be dosed and duration thereof are to be made by a qualified caregiver.

What is claimed is:

1. A pharmaceutical composition comprising of 4-(pyrimidin-2-ylmethyl)-7-(4-(trifluoromethoxy)phenyl)-3,4-dihydrobenzo[f][1,4]oxazepin-5(2H)-one (Compound I), a beta cyclodextrin derivative and a pharmaceutically acceptable excipient or carrier.

2. A pharmaceutical composition consisting essentially of 4-(pyrimidin-2-ylmethyl)-7-(4-(trifluoromethoxy)phenyl)-3,4-dihydrobenzo[f][1,4]oxazepin-5(2H)-one (Compound I), a beta cyclodextrin derivative and a pharmaceutically acceptable excipient or carrier.

3. The pharmaceutical composition according to claim 1 wherein the beta cyclodextrin derivative is Captisol®.

4. The pharmaceutical composition according to claim 1 wherein the beta cyclodextrin derivative is Dexolve-7®.

5. The pharmaceutical composition according to claim 1 comprising a concentrate of about 2.5 mg/mL of Compound (I) in water with about 1 to 12% Captisol®.

6. The pharmaceutical composition according to claim 5 wherein the Captisol® is diluted from a 30% Captisol® concentrate to a centration of between about 1% to about 12% Captisol®.

7. The pharmaceutical composition according to claim 1 comprising a concentrate of about 2.5 mg/mL of Compound (I) in water with about 12% Captisol®.

8. A method of treating a cardiovascular disease comprising administering an intravenous composition comprising Compound (I), a beta-cyclodextrin derivative and a pharmaceutically acceptable excipient or carrier to a human patient in need thereof.

9. The method according to claim 8 wherein the beta cyclodextrin derivative is Captisol® or Dexolve-7®.

10. The method according to claim 8 wherein the beta cyclodextrin derivative is Captisol®.

11. A method of treating a cardiovascular disease comprising administering an intravenous composition consisting essentially of Compound (I) and a beta-cyclodextrin derivative to a human patient in need thereof.

12. The method according to claim 8 wherein the cardiovascular disease is selected from the group consisting of atrial fibrillation, ventricular tachycardia, ventricular fibrillation, LQT syndromes, heart failure, and hypertrophic cardiomyopathy.

13. The method according to claim 12 wherein the LQT syndrome is LQT1, LQT2, or LQT3.

14. Use of an intravenous composition comprising Compound (I) and a beta cyclodextrin derivative for the manufacture of a medicament for the treatment of cardiovascular diseases.

15. The use according to claim 14 wherein the beta cyclodextrin derivative is Captisol® or Dexolve-7®.

16. The use according to claim 14 wherein the beta cyclodextrin derivative is Captisol®.

17. The use according to claim 14 wherein the cardiovascular disease is atrial fibrillation, ventricular tachycardia, ventricular fibrillation, LQT syndromes, heart failure, and hypertrophic cardiomyopathy.

18. The use according to claim 14 wherein the cardiovascular disease is LQT1, LQT2, LQT3 or hypertrophic cardiomyopathy.
